(19) United States
(12) Patent Application Publication
(10) Pub. No.: US 2003/0162827 A1
(43) Pub. Date: Aug. 28, 2003

(54) HMG CoA reductase inhibiting composition, method of preparation thereof and method for competitively inhibiting HMG CoA reductase using such composition

(76) Inventors: Suresh Venkataram, Karnataka (IN); B.K. Mahesh, Karnataka (IN); Nirogi Venkata Satya Ramakrishna, Andhra Pradesh (IN)

Correspondence Address: NIXON & VANDERHYE, PC, 1100 N GLEBE ROAD, 8TH FLOOR, ARLINGTON, VA, 22201-4714, US

(21) Appl. No.: 10/349,172

(22) Filed: Jan. 23, 2003

(30) Foreign Application Priority Data
Jan. 30, 2002 (IN) 84/MUM/2002

(51) Int. Cl. A61K 31/401

(52) U.S. Cl. 514/423

(57) ABSTRACT

The present invention relates to a HMG CoA Reductase inhibiting composition with improved bioavailability. The present invention also relates to a method for preparation of such composition and to the competitive inhibition of HMG CoA Reductase using such composition. The active ingredients are present in a rapidly dissolvable and more solubilized state, thereby enabling sustained and complete solubilization, more rapid dissolution upon administration to a patient, improved absorption and/or bioavailability and also increased flexibility in terms of providing the active ingredient and being capable of administration in the form of a solution, suspension, solid dispersion, solid solution or semi-solid formulation.

### FIELD OF THE INVENTION

The present invention relates to a HMG CoA Reductase inhibiting composition with improved bioavailability. The present invention also relates to a method for preparation of such composition and to the competitive inhibition of HMG CoA Reductase using such composition.

### BACKGROUND OF THE INVENTION

It is known in the art that patients with hyperlipidaemia can be treated with atorvastatin since atorvastatin inhibits the HMG CoA Reductase enzyme, which is involved in cholesterol synthesis.

Atorvastatin is chemically [R-(R*,R*)]-2-(4-FLUOROPHENYL)-β,δ-DIHYDROXY-5-(1-METHYLETHYL)-3-PHENYL-4-[(PHENYLAMINO)CARBONYL]-1H-PYRROLE-1-HEPTANOIC ACID. Atorvastatin calcium, a synthetic HMG-CoA reductase inhibitor, is used for the treatment of hyperlipidaemia and hypercholesterolemia, both of which are risk factors for arteriosclerosis and coronary heart disease. Open dihydroxy carboxylic acid, lactone and various salt forms of atorvastatin have been synthesized.

U.S. Pat. No. 5,273,995 discloses that R-form of the ring opened acid form has surprising inhibition of the biosynthesis of cholesterol. Atorvastatin in its calcium salt form, i.e. [R-(R*,R*)]-2-(4-fluorophenyl)-β,δ-dihydroxy-5-(1-methylethyl)-3-phenyl-4-[(phenylamino)carbonyl]-1H-pyrrole-1-heptanoic acid calcium salt (2:1) having formula 1:

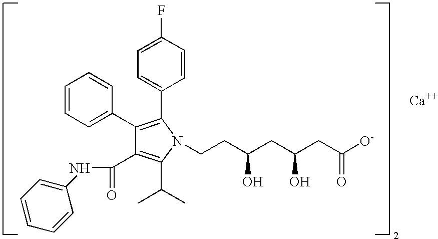

is more suited to formulations and has been recommended as a drug.

U.S. Pat. Nos. 5,003,080; 5,097,045; 5,103,024; 5,124,482; 5,149,837 5,155,251; 5,216,174; 5,245,047; 5,248,793; 5,273,995; 55280,126; 5,298,627; 5,342,952; 5,385,929; 5,397,792; European Patent 409,281; and PCT publication No. 8,907,598 describe various processes and key intermediates for preparing atorvastatin. Atorvastatin is preferably prepared as its calcium salt, i.e. [R-(R*,R*)]-2-(4-fluorophenyl)-β,δ- dihydroxy-5-(1-methylethyl)-3-phenyl-4-[(phenylamino)carbonyl]-1H-pyrrole-1-heptanoic acid calcium salt (2:1).

PCT application, WO 97/03958 and WO 97/03959 disclose novel crystalline forms of atorvastatin calcium designated as Form I, Form II, Form III and Form IV and method for their preparation which provide more favorable filtration and drying characteristics. PCT application, WO 97/03960 and U.S. Pat. No. 6,087,511 describe the procedures for converting the crystalline form of Atorvastatin Calcium to the amorphous form. The process disclosed therein involve dissolving form I atorvastatin calcium in a non-hydroxylic solvent like tetrahydrofuran or a mixture of tetrahydrofuran and toluene. PCT application WO 00/71116 describes the procedure for converting the crystalline form-I by dissolving in a non-hydroxylic solvent like tetrahydrofuran and precipitating atorvastatin calcium amorphous by the addition of non-polar hydrocarbon solvents like, n-hexane, cyclohexane or n-heptane.

A problem faced with atorvastatin calcium is that the aqueous solubility thereof is poor. The dissolution rate of atorvastatin calcium in gastrointestinal fluids is poor due to its poor solubility in water. As a result the bioavailability of atorvastatin calcium is limited, not only by its physical form (amorphous or crystalline) but also by its dissolution rate.

While focus has been given to the preparation of atorvastatin calcium in its amorphous form from the crystalline form in order to improve the bioavailability, sufficient attention has not been paid to improving the bioavailability by enhancing the dissolution rate.

Hydrophobic active ingredients, such as atorvastatin and other statins (such as lovastatin, etc.) suffer from poor aqueous solubility and slow dissolution rate resulting in poor drug delivery. The poor dissolution rate of statin is generally ascribed to the physical form of the statin. In prior art formulations, statins are provided in the form of solids in a carrier. For example, the statin remains in solid form even in a liquid carrier.

Several methods known in the art have attempted to address this problem by trying to enhance in vivo performance. In the manufacture of solid state formulations, it is, for example, known in the art to subject the active ingredient to micronization, or reduce the size in emulsion formulations. However, such methods suffer from problems of both stability and processing, Since the micronized or nanonized active ingredient still possesses high degree of crystallinity, dissolution remains a problem.

U.S. Pat. No. 6,248,363 discloses delivery systems for encapsulated drugs consisting of a wide range of hydrophobic and hydrophilic active ingredients comprising a solid carrier comprising a substrate and preferably an encapsulation coat. The active ingredient is contained in the substrate with hydrophilic and lipophilic surfactants and triglycerides. While, solubilizers such as N-methyl pyrrolidone are indicated as additional ingredients, this patent does not provide any guidance towards enhancing the dissolution of active ingredients such as statins using solubilizers or diluents. On the other hand, solubility of the statins is enhanced using hydrophilic surfactants such as polyethoxylated fatty acids, polyethylene glycol fatty acid esters, and the like. There is also no disclosure or guidance in this U.S. patent as to the formation of solid solutions in order to enhance bioavailability of an active ingredient. On the contrary, in this disclosure, the active ingredient remains in the form of solid in the carrier.

U.S. Pat. No. 6,267,985 discloses the use of surfactants for improving the solubility of triglycerides. According to this disclosure solubility of triglycerides is improved by the use of lipophilic and hydrophilic surfactants and the formulations may contain optionally atorvastatin and other statins as well as pyrrolidones. This patent does not provide any guidance towards enhancing the dissolution of active ingredients such as statins using solubilizers or diluents. Again, in this disclosure, the active ingredient itself remains in the form of solid in the carrier.

U.S. Pat. No. 6,294,192 discloses triglyceride-free pharmaceutical compositions for delivery of hydrophobic therapeutic agents. Compositions of this disclosure include a hydrophobic therapeutic agent and a carrier, where the carrier is formed from a combination of a hydrophilic surfactant and a hydrophobic surfactant. Upon dilution with an aqueous solvent, the composition forms a clear, aqueous dispersion of the surfactants containing the therapeutic agent. This patent also does not provide any guidance towards enhancing the dissolution of active ingredients such as statins using solubilizers or diluents.

Conventional solid dosage forms of hydrophobic active ingredients, such as tablets, of multiparticulates in capsules, often exhibit slow and incomplete dissolution and subsequent absorption. These formulations often show a high propensity for biovariability and food interactions of the active ingredient, resulting in restrictive compliance/labeling requirements. Another problem associated with the prior art solid state formulations is that the active ingredient is still in the form of solid (whether crystalline or amorphous form) in the carrier and as a result, the bioavailability of the active ingredient is low.

One solution to this problem has been to prepare liquid formulations. However, liquid formulations present drug precipitation and packaging challenges. Non-solid formulations are also prone to chemical instability and capsule-shell incompatibility, potential leakage on storage, etc.

Commercially available Lipitor® (Pfizer Inc, USA) an immediate release atorvastatin tablet, shows a bioavailability of only 12% (Martindale, 32^{nd} edition). Another problem is that the product needs to be administered several dosage regimen, such as 10 mg daily, to 20 mg to 80 mg twice a day. Atorvastatin calcium also shows first pass metabolism increasing the amount of drug required for therapeutic effect, thereby increasing the cost of therapy.

Due to the slow dissolution, solid dosage forms often delay the onset of action of some hydrophobic active ingredients. It is therefore essential to provide pharmaceutical compositions and dosage forms, and methods therefor, that do not suffer from the foregoing disadvantages. It is believed that the aforesaid problems of the prior art can be overcome by converting the active ingredient into a solution or a solid dispersion or a solid solution.

### OBJECTS OF THE INVENTION

The main object of the present invention is to provide a pharmaceutical composition wherein the active ingredients are present in a rapidly dissolvable and more solubilized state.

It is another object of the invention to provide a pharmaceutical composition having more rapid dissolution upon administration to a patient.

It is another object of the invention to provide a pharmaceutical composition having more sustained and complete solubilization upon administration to a patient.

It is another object of the invention to provide a pharmaceutical composition capable of delivery of atorvastatin calcium.

It is another object of the invention to provide a pharmaceutical composition having increased flexibility in terms of providing the active ingredient by filling in hard or soft gelatin capsules or formation into molded suppositories.

It is a further object of the invention to provide a formulation capable of administration in form of a solution, suspension, solid dispersion, solid solution or semi-solid formulation.

It is another object of the invention to provide a pharmaceutical composition capable of improved absorption and/or bioavailability of the pharmaceutical active ingredient, atorvastatin calcium.

It is another object of the invention to provide a formulation capable of administration in several routes of administration, whether buccal, sublingual, as a suppository, or oral formulation.

### SUMMARY OF THE INVENTION

The above and other objects of the present invention are achieved by providing a pharmaceutical composition containing atorvastatin calcium for improved delivery thereof to enhance the bioavailability thereof.

The composition of the invention is a synergistic composition evincing unexpected and unknown properties over the aggregate properties of the individual ingredients and is not a simple admixture.

Accordingly, the present invention comprises a HMG CoA Reductase inhibiting composition comprising atorvastatin calcium and a solubilizer in a ratio of 1:1.25 to 1:10 by weight.

The invention also provides a HMG CoA Reductase inhibiting composition comprising atorvastatin calcium and a solubilizer in a ratio of 1:1.25 to 1:10, the balance comprising of a carrier selected from the group consisting of polyethylene glycol 400, polyethylene glycol 1540, polyethylene glycol 4000, polyethylene glycol 6000, bees wax, Gelucires, Labrafil, Labrasol, Labrafac and any mixture thereof.

In another embodiment of the invention the composition contains additional excipients comprising one or more of vehicles such as Miglyol, binders such as Povidone K-30, sweeteners such as aspartame, calcium stearate, flavoring agents to improve palatability such as flavour orange powder, lubricant such as colloidal silicon dioxide, diluent such as pre-gelatinized starch, and any mixture thereof.

The present invention also relates to a process for the preparation of HMG CoA Reductase inhibiting composition comprising atorvastatin calcium and a solubilizer in a ratio of 2:2.5 to 10 by weight, said process comprising mixing said atorvastatin calcium and said solubilizer in any conventional manner.

In one embodiment of the invention, the solubilizer comprises N-methyl pyrrolidone.

In another embodiment of the invention the composition contains additional excipients comprising one or more of carriers such as polyethylene glycol 400, polyethylene glycol 1540, polyethylene glycol 4000, polyethylene glycol 6000, bees wax, Gelucires, Labrafil, Labrasol and Labrafac, vehicles such as Miglyol, binders such as Povidone K-30, sweeteners such as aspartame, calcium stearate, flavoring agents to improve palatability such as flavour orange powder, lubricant such as colloidal silicon dioxide, diluent such as pre-gelatinized starch, and any mixture thereof.

In one embodiment of the invention, the composition comprises atorvastatin calcium in an amount of 2 to 80 mg, N-methyl pyrrolidone in an amount of 5 to 200 mg, and a carrier comprising a mixture of polyethylene glycol 400 in an amount of 0 to 630 mg, polyethylene glycol 1540 in an amount of 0 to 390 mg, polyethylene glycol 4000 in an amount of 0 to 200 mg and polyethylene glycol 6000 in an amount of 0 to 235 mg, said composition being formulated as a dispersion and encapsulated in a gelatin capsule.

In another embodiment of the invention, the composition comprises atorvastatin calcium in an amount of 2 to 80 mg, N-methyl pyrrolidone in an amount of 20 to 200 mg, and a vehicle comprising a mixture of Miglyol in an amount of 0 to 220 mg and polyethylene glycol 400 in an amount of 0 to 480 mg, said composition being formulated as a solution and encapsulated in a gelatin capsule.

In yet another embodiment of the invention, the composition comprises atorvastatin calcium in an amount of 2 to 80 mg, N-methyl pyrrolidone in an amount of 2.5 to 400 mg, and a carrier comprising a mixture of Miglyol in an amount of 0 to 495 mg, polyethylene glycol 400 in an amount of 0 to 530 mg, polyethylene glycol 4000 in an amount of 0 to 120 mg and polyethylene glycol 6000 in an amount of 0 to 10 mg., said composition being formulated as a suspension and encapsulated in a gelatin capsule.

In a further embodiment of the invention, the composition of the invention as prepared is in the form of a suspension, solution, or dispersion contained in a hard or soft gelatin capsule.

In another embodiment of the invention, the composition comprises atorvastatin calcium in an amount of 2 to 80 mg, N-methyl pyrrolidone in an amount of 5 to 200 mg, and carrier comprising a mixture of polyethylene glycol 1540 in an amount of 500 to 775 mg and polyethylene glycol 6000 in an amount of 250 mg, said composition being formulated as a suppository.

In another embodiment, the present invention relates to a process for the preparation of HMG CoA Reductase inhibiting composition comprising atorvastatin calcium and a solubilizer in a ratio of 2:2.5 to 10 by weight, the balance comprising of a carrier selected from the group consisting of polyethylene glycol 400, polyethylene glycol 1540, polyethylene glycol 4000, polyethylene glycol 6000, bees wax, Gelucires, Labrafil, Labrasol, Labrafac and any mixture thereof, said process comprising mixing said atorvastatin calcium, said solubilizer and said carrier in any conventional manner.

The present invention also relates to a method for the treatment of hyperlipidaemia in a patient comprising administering to said patient a composition comprising HMG CoA Reductase inhibiting composition comprising atorvastatin calcium and a solubilizer in a ratio of 2:2.5 to 10 by weight.

In another embodiment of the invention, the composition is administered to said patient in the form of a suspension, solution or dispersion contained in hard or soft gelatin capsule, a tablet or in the form of a suppository.

In one embodiment of the invention, the solubilizer comprises N-methyl pyrrolidone.

In another embodiment of the invention, the composition may comprise additional excipients comprising one or more of carriers such as polyethylene glycol 400, polyethylene glycol 1540, polyethylene glycol 4000, polyethylene glycol 6000, bees wax, Gelucires, Labrafil, Labrasol and Labrafac, vehicle such as Miglyol, binders such as Povidone K-30, sweeteners such as aspartame, calcium stearate, flavoring agents to improve palatability such as flavour orange powder, lubricant such as colloidal silicon dioxide, diluent such as pregelatinised starch, and any mixture thereof.

In one embodiment of the invention, the composition comprises atorvastatin calcium in an amount of 2 to 80 mg, N-methyl pyrrolidone in an amount of 5 to 200 mg, and a carrier comprising a mixture of polyethylene glycol 400 in an amount of 0 to 630 mg, polyethylene glycol 1540 in an amount of 0 to 390 mg, polyethylene glycol 4000 in an amount of 0 to 200 mg and polyethylene glycol 6000 in an amount of 0 to 235 mg, said composition being formulated as a dispersion and encapsulated in a gelatin capsule.

In another embodiment of the invention, the composition comprises atorvastatin calcium in an amount of 2 to 80 mg, N-methyl pyrrolidone in an amount of 20 to 200 mg, and a carrier comprising a mixture of Miglyol in an amount of 0 to 220 mg and polyethylene glycol 400 in an amount of 0 to 480 mg, said composition being formulated as a solution and encapsulated in a gelatin capsule.

In yet another embodiment of the invention, the composition comprises atorvastatin calcium in an amount of 2 to 80 mg, N-methyl pyrrolidone in an amount of 2.5 to 400 mg, and a carrier comprising a mixture of Miglyol in an amount of 0 to 495 mg, polyethylene glycol 400 in an amount of 0 to 530 mg, polyethylene glycol 4000 in an amount of 0 to 120 mg and polyethylene glycol 6000 in an amount of 0 to 10 mg., said composition being formulated as a suspension and encapsulated in a gelatin capsule.

In yet another embodiment of the invention, the composition comprises atorvastatin calcium in an amount of 2 to 20 mg, N-methyl pyrrolidone in an amount of 5 to 50 mg, and a carrier comprising a mixture of polyethylene glycol 1540 in an amount of 700 to 775 mg and polyethylene glycol 6000 in an amount of 250 mg, said composition being formulated as a suppository.

### DETAILED DESCRIPTION OF THE INVENTION

Atorvastatin—chemically [R-(R*,R*)]-2-(4-FLUOROPHENYL)-β,δ-DIHYDROXY-5-(1-METHYLETHYL)-3-PHENYL-4-[(PHENYLAMINO)CARBONYL]-1H-PYRROLE-1-HEPTANOIC ACID is a statin and is useful in the treatment of hyperlipidaemia and hypercholesterolemia, both of which are risk factors for arteriosclerosis and coronary heart disease since it is a synthetic HMG-CoA reductase inhibitor. Open dihydroxy carboxylic acid, lactone and various salt forms of atorvastatin have been synthesized.

Atorvastatin is preferably prepared as its calcium salt, i.e. [R-(R*,R*)]-2-(4-fluorophenyl)-β,δ-dihydroxy-5-(1-methylethyl)-3-phenyl-4-[(phenylamino)carbonyl]-1H-pyrrole-1-heptanoic acid calcium salt (2:1). However, one of the problems of prior art formulations available commercially such as Lipitor®, is that the bioavailability of the drug is poor. Due to the poor dissolution rate of atorvastatin calcium in aqueous media such as gastrointestinal juices, the bioavailability of the formulation is less, thereby requiring higher dosage levels to be given to a patient. This results in the consequent disadvantage of enhanced side effects on the patients due to atorvastatin calcium excess. It is reported that the currently available form of immediate release tablets such as Lipitor® (Pfizer Inc., USA) show bioavailability of only 12% (Martindale, 32^{nd} edition).

A problem with atorvastatin calcium is that it exhibits first pass metabolism. The problems of first pass metabolism and poor dissolution rate are not addressed in prior art formulations. Attempts to overcome the disadvantage of first pass metabolism and low bioavialability by selecting buccal and sublingual formulations do not show positive results.

The present invention resides in the realization that a careful and judicious selection of ingredients will ensure adequate solubilization of atorvastatin calcium resulting in better bioavailability with lesser dosage being required. The use of the formulations of the invention for treating patients with hyperlipidaemia and other indications reduces the cost of therapy, reduces exposure to the drug and therefore minimizes side effects due to atorvastatin calcium. The formulation itself comprises of essentially atorvastatin calcium and a solubilizer such as N-methylpyrrolidone, with the active ingredient atorvastatin being dissolved in the solubilizer. The active ingredient can be dissolved in the solubilizer by any conventional method such as sonication.

The dosage forms of the statin atorvastatin calcium formulated in the invention enhance bioavailability due to the presence of atorvastatin in a finely dispersed or dissolved state in the matrix of the solubilizer and carrier. Thus while the formulation is in the form of a solid solution of the active ingredient in the solubilizer, the formulations can be provided in the form of solutions, solid dispersions or suspensions in conventional dispensing means such as gelatin capsules (soft or hard). The formulations can also be tabletted by any conventional tabletting method or provided as a suppository. In particular, a careful selection of atorvastatin calcium and a carrier, which helps solubilise atorvastatin calcium, enhances the dissolution rate of the end use formulation ensuring immediate release.

The present invention comprises a HMG CoA Reductase inhibiting composition comprising atorvastatin calcium in an amount of 2 to 80 mg and a carrier in an amount of 2.5 to 400 mg. The carrier can comprise a solubilizer such as N-methyl pyrrolidone (Pharmasolve®) or a diluent such as lactose or dicalcium phosphate dihydrate. Conventional additives such as carriers, sweeteners, flavouring agents, palatability enhancers, binders, surfactants can also be added to the formulation.

The formulation of the invention generally comprises atorvastatin calcium in an amount of 2 to 80 mg, N-methyl pyrrolidone in an amount of 5 to 200 mg, and a carrier comprising a mixture of polyethylene glycol 400 in an amount of 0 to 630 mg, polyethylene glycol 1540 in an amount of 0 to 390 mg, polyethylene glycol 4000 in an amount of 0 to 200 mg and polyethylene glycol 6000 in an amount of 0 to 235 mg, with the composition being formulated as a dispersion and encapsulated in a gelatin capsule. While atorvastatin calcium itself is insoluble in the polyethylene glycol, the addition of polyethylene glycol to the formulation comprising atorvastatin calcium in the solubilizer results in the change of physical state of the formulation from liquid solution to solid or semi solid state thus providing a wider choice of the mode of delivery, whether solution, dispersion, suspension, semi solid or solid solution due to the addition of carriers such as polyethylene glycol, without requiring additional amounts of other ingredient. As a result, the formulation of the invention can be provided in when filled in hard or soft gelatin capsules or even in the form of molded suppositories.

The composition also comprises atorvastatin calcium in an amount of 2 to 80 mg, N-methyl pyrrolidone in an amount of 20 to 200 mg, and a carrier comprising a mixture of Miglyol in an amount of 0 to 220 mg and polyethylene glycol 400 in an amount of 0 to 480 mg, the composition being formulated as a solution and encapsulated in a gelatin capsule.

Preferably, the composition comprises atorvastatin calcium in an amount of 2 to 80 mg, N-methyl pyrrolidone in an amount of 2.5 to 400 mg, and a carrier comprising a mixture of Miglyol in an amount of 0 to 495 mg, polyethylene glycol 400 in an amount of 0 to 530 mg, polyethylene glycol 4000 in an amount of 0 to 120 mg and polyethylene glycol 6000 in an amount of 0 to 10 mg, the composition being formulated as a suspension and encapsulated in a gelatin capsule.

The bioavailability of atorvastatin calcium in the form of a buccal tablet formulation without either N-methylpyrrolidone or polyethylene glycol carrier was tested. The buccal tablet comprised of atorvastatin calcium in an amount of 2 to 20 mg, dicalcium phosphate dihydrate in an amount of 120 to 140 mg, Povidone K-30 in an amount of 3 mg, aspartame in an amount of 1 to 4 mg, calcium stearate in an amount of 0.5 mg and colloidal silicon dioxide in an amount of 0.5 mg. The ingredients were mixed thoroughly and then tabletted in any conventional manner. Preferably, the tablet form of the composition comprises atorvastatin calcium in an amount of 2 to 20 mg, lactose in an amount of 90 to 110 mg, pregelatinised starch in an amount of 5 mg, aspartame in an amount of 1 to 4 mg, flavour orange powder in an amount of 5 mg, calcium stearate in an amount of 0.5 mg and colloidal silicon dioxide in an amount of 0.5 mg. Comparison with even conventional Lipitor® showed that the performance of the buccal or sublingual tablet without N-methylpyrrolidone and/or polyethylene glycol did not enhance bioavailability.

The composition of the invention provides ease of formulation into any form desired, whether hard or soft gelatin capsule or suppository, and is therefore independent of the route of administration. To prepare the solution form of the formulation to be stored in a capsule whether soft or hard gelatin, atorvastatin calcium and N-methyl pyrrolidone in the desired amounts are taken and sonicated for a period of about 10 minutes to obtain an atorvastatin solution in the solubilizer. Miglyol is then added and the mixture further sonicated for a period of about 1 minute to obtain the final formulation. The final formulation is then filled into soft or hard gelatin capsules and sealed using aqueous gelatin solution. To obtain a suspension form, the lumps of atorvastatin are sonicated till the lumps break into fine particles and then mixed with N-methyl pyrrolidone. Miglyol is then added and sonicated for 1 minute with the filling and sealing in gelatin capsules being done in any conventional manner.

To obtain a solid dispersion, the atorvastatin solution in N-methyl pyrrolidone is prepared as explained above. Polyethylene glycol is added till the mixture forms a solid dispersion. This is then sonicated for about 1 minute and allowed to cool to a temperature of about 37° C.±2° C. Filling in the gelatin capsules is done in the same manner as given above.

Suppositories are formed by preparing the atorvastatin solution in N-methyl pyrrolidone as explained above, adding polyethylene glycol with high molecular weight, sonicating for a period of about 1 minute and then filling the mixture in a suppository mold and allowing to cool to a temperature of about 20 to 25° C.

The invention will be explained with reference to the accompanying examples, which are illustrative and should not be construed as limiting the scope of the invention.

### Formation of Solution Form of Composition

### EXAMPLE 1

Atorvastatin calcium, N-methyl pyrrolidone and polyethylene glycol 400 were mixed and formulated as a solution. The solution was then filled in a hard gelatin capsule such that the capsule contained Atorvastatin calcium in an amount of 10 mg, N-methyl pyrrolidone in an amount of 100 mg and polyethylene glycol 400 in an amount of 400 mg.

### EXAMPLE 2

Atorvastatin calcium, N-methyl pyrrolidone and polyethylene glycol 400 were mixed and formulated as a solution. The solution was then filled in a hard gelatin capsule such that the capsule contained Atorvastatin calcium in an amount of 2 mg, N-methyl pyrrolidone in an amount of 20 mg and polyethylene glycol 400 in an amount of 480 mg.

### EXAMPLE 3

Atorvastatin calcium, N-methyl pyrrolidone, polyethylene glycol 400 and polyethylene glycol 6000 were mixed and formulated as a solid dispersion. The dispersion was then filled in a hard gelatin capsule such that the capsule contained Atorvastatin calcium in an amount of 10 mg, N-methyl pyrrolidone in an amount of 100 mg, polyethylene glycol 400 in an amount of 530 mg and polyethylene glycol 6000 in an amount of 10 mg.

### EXAMPLE 4

### Bioavailability Study

The single dose oral bioavailability of the capsules prepared in Examples 1 to 3 were tested vis á vis commercially available Lipitor®. The test design was an open label, single dose, relative bioavailabihity study using three subjects for each formulation.

Results

It was noted that the relative bioavailability of the solid dispersion of Example 3 was 113%, followed by the solution of Example 1 at 88% and then Example 2 solution at 23% compared to Lipitor®. Example 2 dosage was one fifth the dose of Lipitor®. The Cₘₐₓ of solid dispersion of Example 3 was the highest at 4.39 (SEM 1.52) ng/ml as compared to 3.02 (SEM 0.33) ng/ml of Lipitor®.

### EXAMPLE 5

Atorvastatin calcium, N-methyl pyrrolidone, polyethylene glycol 400 and polyethylene glycol 4000 were mixed and formulated as a solid dispersion. The dispersion was then filled in a hard gelatin capsule such that the capsule contained Atorvastatin calcium in an amount of 10 mg, N-methyl pyrrolidone in an amount of 100 mg, polyethylene glycol 400 in an amount of 490 mg and polyethylene glycol 4000 in an amount of 50 mg.

### EXAMPLE 6

### Bioavailability Study

The objective of this study was to compare a 10 mg and a 20 mg (10 mg×2) dose oral bioavailability of the formulation of Example 5 vis a vis Lipitor® as the reference. The single dose oral bioavailability of the capsule prepared in Example 5 was tested vis á vis commercially available Lipitor® in one 10 mg dose and a 20 mg (10 mg×2) dose. The test design was an open label, single dose, cross-over relative bioavailability study using twelve subjects for 10 mg dose comparison and six subjects for the 10 mg×2 dose comparison.

Results

It was noted that the relative bioavailability of the solid dispersion of Example 5 was 111% for the 10 mg dose and 124.4% for the 20 mg dose as compared to corresponding doses of Lipitor®. The Cₘₐₓ of the 10 mg dose was 7.96 (SEM 1.07) ng/ml and 16.77 (SEM 7.1) ng/ml for the 20 mg dose of Example 5 formulations, as compared to 4.82 (SEM 0.75) ng/ml and 8.66 (SEM 2.3 5) ng/ml for the 10 mg and 20 mg doses respectively of Lipitor®.

### EXAMPLE 7

A buccal tablet formulation was prepared by conventional means such that each tablet contained a 10 mg dose of atorvastatin calcium. The buccal tablet prepared comprises

| | |
|---|---|
| 1. Atorvastatin | 10.0 to 80.0 mg |
| 2. Dicalcium phosphate | 63.0 to 133.0 mg |
| 3. Povidone | 3.0 mg |
| 4. Aspartame | 2.0 mg |
| 5. Calcium stearate | 0.5 mg |
| 6. Colloidal silicon dioxide | 0.5 mg |

### EXAMPLE 8

### Bioavailability Study

The objective of this study was to compare the single dose oral bioavialability of a Buccal tablet (10 mg) prepared in Example 7 vis a vis Lipitor® as the reference. The test design was an open label, random, three treatment two sequence, three period, single dose, cross-over bioequivalence study using six subjects for each formulation.

Results

The relative bioavailability of the Buccal tablet as compared to Lipitor® was found to be 49.17%. The Cₘₐₓ of the Buccal tablet was found to be lower than that of Lipitor®, while the Tₘₐₓ was found to be higher than the reference product.

### EXAMPLE 9

A sublingual tablet formulation was prepared by conventional means such that each tablet contained a 10 mg dose of atorvastatin calcium.

The sublingual tablet as prepared comprised of

| | |
|---|---|
| 1. Atorvastatin | 10.0 to 80.0 mg |
| 2. Lactose | 32.0 to 102.0 mg |
| 3. Pregelatinised starch | 5.0 mg |
| 4. Aspartame | 2.0 mg |
| 5. Calcium stearate | 0.5 mg |
| 6. Colloidal silicon dioxide | 0.5 mg |
| 7. Orange flavor powder | 5.0 mg |

### EXAMPLE 10

### Bioavailability Study

The objective of this study was to compare the single dose oral bioavialability of a sublingual tablet (10 mg) prepared in Example 9 vis a vis Lipitor® as the reference. The test design was an open label, random, three treatment two sequence, three period, single dose, cross-over bioequivalence study using eight subjects for each formulation.

Results

The relative bioavailability of the sublingual tablet as compared to Lipitor® was found to be 66.01%. The Cₘₐₓ of the Buccal tablet was found to be lower than that of Lipitor®, while the Tₘₐₓ was found to be higher than the reference product

We claim:

1. A HMG CoA Reductase inhibiting composition comprising atorvastatin calcium and a solubilizer in a ratio of 2:2.5 to 10 by weight.

2. A HMG CoA Reductase inhibiting composition as claimed in claim 1 wherein the solubilizer comprises N-methyl pyrrolidone.

3. A HMG CoA Reductase inhibiting composition as claimed in claim 1 wherein additional excipients selected from one or more carriers, binders, vehicles, diluents, solubilizers, sweeteners, lubricants and flavouring agents are present.

4. A HMG CoA Reductase inhibiting composition as claimed in claim 3 wherein said carrier is selected from the group comprising polyethylene glycol 400, polyethylene glycol 1540, polyethylene glycol 4000, polyethylene glycol 6000 and any mixture thereof.

5. A HMG CoA Reductase inhibiting composition as claimed in claim 3 wherein said vehicle comprises Miglyol.

6. A HMG CoA Reductase inhibiting composition as claimed in claim 3 wherein said binder comprises Povidone K-30.

7. A HMG CoA Reductase inhibiting composition as claimed in claim 3 wherein said sweetener is selected from aspartame and calcium stearate.

8. A HMG CoA Reductase inhibiting composition as claimed in claim 3 wherein said flavoring agent comprises flavour orange powder.

9. A HMG CoA Reductase inhibiting composition as claimed in claim 3 wherein said lubricant comprises colloidal silicon dioxide.

10. A HMG CoA Reductase inhibiting composition as claimed in claim 3 wherein said diluent comprises pregelatinised starch.

11. A HMG CoA Reductase inhibiting composition as claimed in claim 3 wherein the composition comprises atorvastatin calcium in an amount of 2 to 80 mg, N-methyl pyrrolidone in an amount of 5 to 200 mg, and a carrier comprising a mixture of polyethylene glycol 400 in an amount of 0 to 630 mg, polyethylene glycol 1540 in an amount of 0 to 390 mg, polyethylene glycol 4000 in an amount of 0 to 200 mg and polyethylene glycol 6000 in an amount of 0 to 235 mg, said composition being formulated as a dispersion and encapsulated in a gelatin capsule.

12. A HMG CoA Reductase inhibiting composition as claimed in claim 3 wherein the composition comprises atorvastatin calcium in an amount of 2 to 80 mg, N-methyl pyrrolidone in an amount of 20 to 200 mg, and a vehicle comprising a mixture of Miglyol in an amount of 0 to 220 mg and polyethylene glycol 400 in an amount of 0 to 480 mg, said composition being formulated as a solution and encapsulated in a gelatin capsule.

13. A HMG CoA Reductase inhibiting composition as claimed in claim 3 wherein the composition comprises atorvastatin calcium in an amount of 2 to 80 mg, N-methyl pyrrolidone in an amount of 2.5 to 400 mg, and a carrier comprising a mixture of Miglyol in an amount of 0 to 495 mg, polyethylene glycol 400 in an amount of 0 to 530 mg, polyethylene glycol 4000 in an amount of 0 to 120 mg and polyethylene glycol 6000 in an amount of 0 to 10 mg, said composition being formulated as a suspension and encapsulated in a gelatin capsule.

14. A HMG CoA Reductase inhibiting composition as claimed in claim 1 wherein the composition of the invention as prepared is in the form of a suspension, solution, or dispersion contained in a hard or soft gelatin capsule.

15. A HMG CoA Reductase inhibiting composition as claimed in claim 3 wherein the composition comprises atorvastatin calcium in an amount of 2 to 20 mg, N-methyl pyrrolidone in an amount of 5 to 50 mg, and carrier comprising a mixture of polyethylene glycol 1540 in an amount of 700 to 775 mg and polyethylene glycol 6000 in an amount of 250 mg, said composition being formulated as a suppository.

16. A HMG CoA Reductase inhibiting composition comprising atorvastatin calcium and a solubilizer in a ratio of 1:1.25 to 1:10, the balance comprising of a carrier selected from the group consisting of polyethylene glycol 400, polyethylene glycol 1540, polyethylene glycol 4000, polyethylene glycol 6000, bees wax, Gelucires, Labrafil, Labrasol, Labrafac and any mixture thereof.

17. A HMG CoA Reductase inhibiting composition as claimed in claim 16 wherein the composition contains additional excipients comprising one or more of vehicles such as Miglyol, binders such as Povidone K-30, sweeteners such as aspartame, calcium stearate, flavoring agents to improve palatability such as flavour orange powder, lubricant such as colloidal silicon dioxide, diluent such as pre-gelatinized starch, and any mixture thereof.

18. HMG CoA Reductase inhibiting composition as claimed in claim 16 wherein the solubilizer comprises N-methyl pyrrolidone.

19. A HMG CoA Reductase inhibiting composition as claimed in claim 17 wherein said vehicle comprises Miglyol.

20. A HMG CoA Reductase inhibiting composition as claimed in claim 17 wherein said binder comprises Povidone K-30.

21. A HMG CoA Reductase inhibiting composition as claimed in claim 17 wherein said sweetener is selected from aspartame and calcium stearate.

22. A HMG CoA Reductase inhibiting composition as claimed in claim 17 wherein said flavoring agent comprises flavour orange powder.

23. A HMG CoA Reductase inhibiting composition as claimed in claim 17 wherein said lubricant comprises colloidal silicon dioxide.

24. A HMG CoA Reductase inhibiting composition as claimed in claim 17 wherein said diluent comprises pregelatinised starch.

25. A HMG CoA Reductase inhibiting composition as claimed in claim 17 wherein the composition comprises atorvastatin calcium in an amount of 2 to 80 mg, N-methyl pyrrolidone in an amount of 5 to 200 mg, and a carrier comprising a mixture of polyethylene glycol 400 in an amount of 0 to 630 mg, polyethylene glycol 1540 in an amount of 0 to 390 mg, polyethylene glycol 4000 in an amount of 0 to 200 mg and polyethylene glycol 6000 in an amount of 0 to 235 mg., said composition being formulated as a dispersion and encapsulated in a gelatin capsule.

26. A HMG CoA Reductase inhibiting composition as claimed in claim 17 wherein the composition comprises atorvastatin calcium in an amount of 2 to 80 mg, N-methyl pyrrolidone in an amount of 20 to 200 mg, and a vehicle comprising a mixture of Miglyol in an amount of 0 to 220 mg and polyethylene glycol 400 in an amount of 0 to 480 mg, said composition being formulated as a solution and encapsulated in a gelatin capsule.

27. A HMG CoA Reductase inhibiting composition as claimed in claim 17 wherein the composition comprises atorvastatin calcium in an amount of 2 to 80 mg, N-methyl pyrrolidone in an amount of 2.5 to 400 mg, and a carrier comprising a mixture of Miglyol in an amount of 0 to 495 mg, polyethylene glycol 400 in an amount of 0 to 530 mg, polyethylene glycol 4000 in an amount of 0 to 120 mg and polyethylene glycol 6000 in an amount of 0 to 10 mg, said composition being formulated as a suspension and encapsulated in a gelatin capsule.

28. A HMG CoA Reductase inhibiting composition as claimed in claim 17 wherein the composition of the invention as prepared is in the form of a suspension, solution, of dispersion contained in a hard or soft gelatin capsule.

29. A HMG CoA Reductase inhibiting composition as claimed in claim 17 wherein the composition comprises atorvastatin calcium in an amount of 2 to 20 mg, N-methyl pyrrolidone in an amount of 5 to 50 mg, and carrier comprising a mixture of polyethylene glycol 1540 in an amount of 700 to 775 mg and polyethylene glycol 6000 in an amount of 250 mg. said composition being formulated as a suppository.

30. A process for the preparation of ERG CoA Reductase inhibiting composition comprising atorvastatin calcium and a solubilizer in a ratio of 2:2.5 to 10 by weight, said process comprising mixing said atorvastatin calcium and said solubilizer in any conventional manner.

31. A process as claimed in claim 30 wherein the solubilizer comprises N-methyl pyrrolidone.

32. A process as claimed in claim 30 wherein one or more of additional excipients comprising one or more of carriers, vehicles, binders, sweeteners, flavoring agents to improve palatability, lubricant, and diluent, and any mixture thereof are added to the composition, to obtain transition from solid to semi-solid to suspension to liquid formulation.

33. A process as claimed in claim 32 wherein said vehicle comprises Miglyol.

34. A process as claimed in claim 32 wherein said binder comprises Povidone K-30.

35. A process as claimed in claim 32 wherein said sweetener is selected from aspartame and calcium stearate.

36. A process as claimed in claim 30 wherein said flavoring agent comprises flavour orange powder.

37. A process as claimed in claim 32 wherein said lubricant comprises colloidal silicon dioxide.

38. A process as claimed in claim 32 wherein said diluent comprises pregelatinised starch.

39. A process as claimed in claim 32 wherein the composition comprises atorvastatin calcium in an amount of 2 to 80 mg, N-methyl pyrrolidone in an amount of 5 to 200 mg, and a carrier comprising a mixture of polyethylene glycol 400 in an amount of 0 to 630 mg, polyethylene glycol 1540 in an amount of 0 to 390 mg, polyethylene glycol 4000 in an amount of 0 to 200 mg and polyethylene glycol 6000 in an amount of 0 to 235 mg, said composition being formulated as a dispersion and encapsulated in a gelatin capsule.

40. A process as claimed in claim 32 wherein the composition comprises atorvastatin calcium in an amount of 2 to 80 mg, N-methyl pyrrolidone in an amount of 20 to 200 mg, and a carrier comprising a mixture of Miglyol in an amount of 0 to 220 mg and polyethylene glycol 400 in an amount of 0 to 480 mg, said composition being formulated as a solution and encapsulated in a gelatin capsule.

41. A process as claimed in claim 32 wherein the composition comprises atorvastatin calcium in an amount of 2 to 80 mg, N-methyl pyrrolidone in an amount of 2.5 to 400 mg, and a carrier comprising a mixture of Miglyol in an amount of 0 to 495 mg, polyethylene glycol 400 in an amount of 0 to 530 mg, polyethylene glycol 4000 in an amount of 0 to 120 mg and polyethylene glycol 6000 in an amount of 0 to 10 mg., said composition being formulated as a suspension and encapsulated in a gelatin capsule.

42. A process as claimed in claim 32 wherein the composition of the invention as prepared is in the form of a suspension, solution, or dispersion contained in a hard or soft gelatin capsule.

43. A process as claimed in claim 32 wherein the composition comprises atorvastatin calcium in an amount of 2 to 20 mg, N-methyl pyrrolidone in an amount of 5 to 50 mg, and a carrier comprising a mixture of polyethylene glycol 1540 in an amount of 700 to 775 mg and polyethylene glycol 6000 in an amount of 250 mg, said composition being formulated as a suppository.

44. A process for the preparation of HMG CoA Reductase inhibiting composition comprising atorvastatin calcium and a solubilizer in a ratio of 1:1.25 to 1:10 by weight, the balance comprising of a carrier selected from the group consisting of polyethylene glycol 400, polyethylene glycol 1540, polyethylene glycol 4000, polyethylene glycol 6000, bees wax, Gelucires, Labrafil, Labrasol, Labrafac and any mixture thereof, said process comprising mixing said atorvastatin calcium, said solubilizer and said carrier in any conventional manner.

45. A process as claimed in claim 44 wherein the solubilizer comprises N-methyl pyrrolidone.

46. A process as claimed in claim 44 wherein additional excipients comprising one or more of vehicles, binders, sweeteners, flavoring agents to improve palatability, lubricant, and diluent, and any mixture thereof are added to the composition.

47. A process as claimed in claim 46 wherein said vehicle comprises Miglyol.

48. A process as claimed in claim 46 wherein said binder comprises Povidone K-30.

49. A process as claimed in claim 46 wherein said sweetener is selected from aspartame and calcium stearate.

50. A process as claimed in claim 46 wherein said flavoring agent comprises flavour orange powder.

51. A process as claimed in claim 46 wherein said lubricant comprises colloidal silicon dioxide.

52. A process as claimed in claim 46 wherein said diluent comprises pregelatinised starch.

53. A process as claimed in claim 46 wherein the composition comprises atorvastatin calcium in an amount of 2 to 80 mg, N-methyl pyrrolidone in an amount of 5 to 200 mg, and a carrier comprising a mixture of polyethylene glycol 400 in an amount of 0 to 630 mg, polyethylene glycol 1540 in an amount of 0 to 390 mg, polyethylene glycol 4000 in an amount of 0 to 200 mg and polyethylene glycol 6000 in an amount of 0 to 235 mg, said composition being formulated as a dispersion and encapsulated in a gelatin capsule.

54. A process as claimed in claim 46 wherein the composition comprises atorvastatin calcium in an amount of 2 to 80 mg, N-methyl pyrrolidone in an amount of 20 to 200 mg, and a carrier comprising a mixture of Miglyol in an amount of 0 to 220 mg and polyethylene glycol 400 in an amount of 0 to 480 mg, said composition being formulated as a solution and encapsulated in a gelatin capsule.

55. A process as claimed in claim 46 wherein the composition comprises atorvastatin calcium in an amount of 2 to 80 mg, N-methyl pyrrolidone in an amount of 2.5 to 400 mg, and a carrier comprising a mixture of Miglyol in an amount of 0 to 495 mg, polyethylene glycol 400 in an amount of 0 to 530 mg, polyethylene glycol 4000 in an amount of 0 to 120 mg and polyethylene glycol 6000 in an amount of 0 to 10 mg, said composition being formulated as a suspension and encapsulated in a gelatin capsule.

56. A process as claimed in claim 46 wherein the composition of the invention as prepared is in the form of a suspension, solution, or dispersion contained in a hard or soft gelatin capsule.

57. A process as claimed in claim 46 wherein the composition comprises atorvastatin calcium in an amount of 2 to 20 mg, N-methyl pyrrolidone in an amount of 5 to 50 mg, and a carrier comprising a mixture of polyethylene glycol 1540 in an amount of 700 to 775 mg and polyethylene glycol 6000 in an amount of 250 mg, said composition being formulated as a suppository.

58. A method for the treatment of hyperlipidaemia in a patient comprising administering to said patient a composition comprising HMG CoA Reductase inhibiting composition comprising atorvastatin calcium and a solubilizer in a ratio of 1:1.25 to 1:10 by weight.

59. A method as claimed in claim 58 wherein the composition is administered to said patient in the form of a suspension, solution or dispersion contained in hard or soft gelatin capsule, a tablet or in the form of a suppository.

60. A method as claimed in claim 58 wherein the solubilizer comprises N-methyl pyrrolidone.

61. A method as claimed in claim 58 wherein the composition comprises additional excipients comprising one or more of carriers such as polyethylene glycol 400, polyethylene glycol 1540, polyethylene glycol 4000, polyethylene glycol 6000, bees wax, Gelucires, Labrafil, Labrasol and Labrafac, vehicle such as Miglyol, binders such as Povidone K-30, sweeteners such as aspartame, calcium stearate, flavoring agents to improve palatability such as flavour orange powder, lubricant such as colloidal silicon dioxide, diluent such as pregelatinised starch, and any mixture thereof.
